(19) United States
(12) Patent Application Publication
(10) Pub. No.: US 2007/0088214 A1
(43) Pub. Date: Apr. 19, 2007

(54) Implantable physiologic monitoring system

(75) Inventors: Allan Shuros, St. Paul, MN (US); Rodney Salo, Fridley, MN (US); Michael Kane, Lake Elmo, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers Inc.

(21) Appl. No.: 11/250,928

(22) Filed: Oct. 14, 2005

(51) Int. Cl. A61B 8/00

(52) U.S. Cl. 600/437

(57) ABSTRACT

An ultrasonic implantable device includes an ultrasonic sensor having a plurality of transducers. The sensor is configured for mounting to a vessel wall. A first of the transducers directs sound waves in a direction at least partially upstream or downstream in the vessel. A second of the transducers directs sound waves in a radial direction through an interior of the vessel against a sidewall of the vessel. The sensor monitors a change in frequency of the sound waves from the first transducer to determine a fluid velocity in the vessel. The sensor also monitors a reflection time of the sound waves from the second transducer that return from the sidewall to determine an internal diameter of the vessel. The determined fluid velocity and vessel diameter can be used to determine a volumetric flow rate of the fluid in the vessel.

### TECHNICAL FIELD

The present invention generally relates to implantable physiologic devices and more specifically relates to implantable ultrasonic devices.

### BACKGROUND

A wide variety of implantable medical devices (IMDs) have been developed for the purpose of monitoring and managing physiological parameters in a patient. Implantable pacemakers and cardioverter-defibrillators (ICDs) are example IMDs that have been effective in not only monitoring but also providing treatment to the patient automatically based on monitored conditions. Some implantable diagnostic and monitoring systems monitor patients in their home environments, providing treating physicians with more complete information about their patients changing physiologic conditions. Some advanced patient management systems provide for automated downloading and uploading of information to the implanted device through wireless communication that occurs manually or automatically on a periodic basis thereby providing desired information for the treating physician as well as for the patient.

One important physiological parameter that can be monitored and then used for calculating a variety of other parameters and determining optimal treatment scenarios is the cardiac stroke volume (e.g., the amount of blood expelled by the heart with every contraction) and combined with heart rate (HR) to determine cardiac output. An accurate assessment of stroke volume can be useful for the purpose of determining, for example, whether there is enough blood flowing in the patient's body to sustain patient consciousness, whether the heart is pumping efficiently given a monitored contraction rate, and whether such conditions as decompensation exist given a monitored contractility parameter and contraction rate. Relative changes over time in stroke volume and cardiac output may be indicative of changes in cardiac physiology and pathology.

Obtaining an accurate stroke volume can be very difficult due to the many variables that modulate the circulatory system. Various portions of the heart and connecting vessels expand and contract over time in response to pressure variations produced by the heart. Circulatory dynamics including, for example, preload, filling volume, and resistances can vary with internal and external factors such as age, stress, ambient temperature, humidity, etc. to influence cardiac output. Also, the rate of blood flow will vary significantly depending on the activity in which the patient is involved (e.g., exercise vs. sleeping).

### SUMMARY

The present invention relates generally to implantable physiologic monitoring devices. More particularly, the present disclosure relates to implantable ultrasonic devices for use in monitoring cardiac and other physiologic conditions/parameters. A benefit of the embodiments disclosed herein is an accurate assessment of the stroke volume and cardiac output using an in vivo method. This technology may be used for optimization of such therapies as, for example, pharmacological, pacing resynchronization, pacing rate and other interchamber pacing timing variables, etc.

One aspect of the present disclosure relates to an ultrasonic implantable device that includes an ultrasonic sensor having a plurality of electro-acoustic transducers. The sensor is configured for mounting on the inner surface of a vessel wall. A first of the transducers directs sound waves in a direction at least partially upstream or downstream in the vessel. A second of the transducers directs sound waves in a transverse direction through an interior of the vessel against a sidewall of the vessel. The sensor monitors a change in frequency of the sound waves from the first transducers to determine a fluid velocity in the vessel. The sensor also monitors a reflection time of the sound waves from the second transducers that return from the sidewall to determine an internal diameter of the vessel. The determined fluid velocity and vessel diameter can be used to calculate a volumetric flow rate of the fluid in the vessel.

Another aspect of the present disclosure relates to a method of monitoring heart performance. The method includes mounting an ultrasonic sensor having a plurality of transducers to a vessel wall of a first vessel and directing sound waves from the sensor upstream or downstream in the first vessel to determine a velocity of blood flow in the first vessel. The method also includes directing sounds waves from the sensor across the first vessel towards an opposing sidewall of the first vessel to determine a diameter of the first vessel and determining blood stroke volume of the heart based on the determined blood flow velocity and the diameter of the first vessel.

A still further aspect of the invention relates to a method of monitoring blood flow in a first vessel. The method includes mounting an ultrasonic sensor having a plurality of transducers to a wall of a second vessel, directing sound waves from the second sensor upstream or downstream in the first vessel to determine a velocity of blood flow in the first vessel, and directing sound waves from the second sensor across an interior of the first vessel to determine a diameter of the first vessel. The volumetric flow of blood through the first vessel can then be determined based on the blood flow velocity and vessel diameter in the first vessel.

The above summary is not intended to describe each disclosed embodiment or every implementation of the present invention. The figures and the detailed description that follow further describe these embodiments.

### BRIEF DESCRIPTION OF THE DRAWINGS

Aspects of the invention may be more completely understood in consideration of the following detailed description of various embodiments of the invention in connection with the accompanying drawings, in which:

FIG. 1 illustrates an example ultrasonic device positioned on an interior sidewall of a vessel;

FIG. 2 illustrates another example ultrasonic device positioned on an exterior sidewall of a vessel on a side opposite a second vessel;

FIG. 3 illustrates another example ultrasonic device positioned on an exterior sidewall of a vessel on a side adjacent to a second vessel;

FIG. 4 illustrates another example ultrasonic device positioned on an interior sidewall of a vessel on a side adjacent to a second vessel;

FIG. 5 illustrates an example sensor having a phased array of transducers;

FIG. 6 illustrates a pair of sensors positioned adjacent to each other and each sensor having a plurality of transducers for either transmitting or receiving signals;

FIG. 7 illustrates another example sensor having transducers fixed at angled positions relative to each other;

FIG. 8 illustrates an example implantable medical device (IMD) in wired communication with an ultrasonic device in a patient;

FIG. 9 is a flow diagram illustrating example steps of monitoring blood flow according to principles of the disclosed invention; and

FIG. 10 is a flow diagram illustrating example steps of monitoring heart performance according to principles of the disclosed invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present disclosure relates generally to implantable physiologic monitoring devices. More particularly, the present disclosure relates to implantable ultrasonic devices for use in monitoring cardiac and other physiologic conditions/parameters.

The term “patient” is used herein to mean any individual wherein a monitoring device is implanted. The term “caregiver” is used herein to mean any provider of services, such as health care providers including, but not limited to, nurses, doctors, and other health care provider staff.

One aspect of the present disclosure relates to the use of implantable ultrasonic devices that are used to measure and monitor stroke volume and cardiac output of the patient's heart. The ultrasonic device may be mounted either to an interior wall or an exterior wall of a vessel. The vessel may be positioned in close proximity to the heart. Proximity of the ultrasonic device to the heart may affect accuracy of the assessed stroke volume. The ultrasonic device may be configured to determine, with the use of ultrasound, the velocity of blood flow in the vessel as well as the internal diameter or other dimensions of the vessel. The stroke volume of the heart can be determined in whole or in part based on calculations using the flow velocity and vessel diameter. The diameter measurement can be obtained at various rates compared to the cardiac cycle. For instance, the diameter can be measured at a rate much faster than the heart rate, thus giving the diameter value as a function of time over the cardiac cycle. A mathematical operation can be applied to the diameter signal to closely estimate the vessel cross sectional area, which becomes the area signal. The diameter measurement can also be obtained at rates equal to or less than the heart rate. This approach saves energy and reduces complexity, with a concomitant loss of detail.

Stroke volume can be calculated by integrating the velocity signal times the area signal over one cardiac cycle. The calculation can employ the velocity periodicity or the diameter periodicity inherent in the measured parameters to estimate the cardiac cycle length. Alternately, the cardiac cycle length can be obtained via communication with another implantable medical device such as, for example, a defibrillator or pacemaker.

In one example, the vessel to which the ultrasonic device is mounted on an interior sidewall of a major vessel that is in direct fluid flow communication with one of the heart chambers. In another example, the ultrasonic device is mounted on an exterior sidewall of a major vessel that is in direct fluid flow communication with one of the heart chambers. In either embodiment, the ultrasonic device is capable of monitoring fluid flow within the vessel to which it is attached, in another vessel extending in close proximity to the vessel, or in the heart or other organs in close proximity to the vessel. The ultrasonic device can determine vessel and heart chamber diameters, flow rates within the vessels and heart chambers, stroke volume for the heart, and fluid flow volume generally in the vessels. Determining such characteristics of the blood, vessels and heart can be done without physically contacting the blood, vessels and heart when using ultrasound signals generated by the ultrasound device.

In other embodiments, the ultrasonic device may be mounted to a coronary artery or vein or to the pericardial sac surrounding the heart. With these configurations, the ultrasonic device can determine fluid flow through the desired coronary arteries or veins or fluid flow within the heart chambers themselves.

The ultrasonic device may also be mounted to vessels at locations remote from the heart or in vessels that correlate to specific circulatory subsystems. The ultrasonic device may be configured to determine the blood flow rates into or out of specific remotely located vessels or circulatory sub-systems such as, for example, the renal, femoral or hepatic circulatory sub-systems.

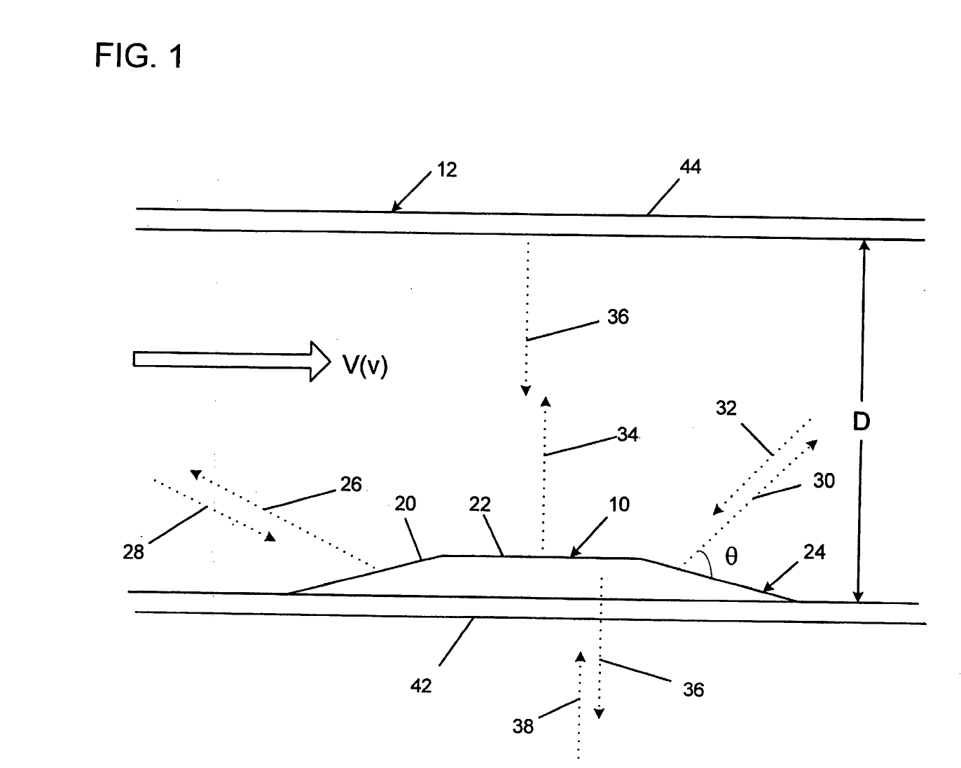

Referring now to FIG. 1, an example implantable ultrasonic device 10 is shown and described with reference to a vessel 12. The vessel 12 has a diameter D and opposing sidewalls 42, 44. A fluid flow V passes through the vessel 12. The vessel has a generally circular cross section radially through the vessel (not shown) wherein the diameter D is substantially the same at all points around the internal circumference of the vessel. The ultrasonic device 110 is mounted to an internal surface of sidewall 42 of a vessel 12.

The ultrasonic device 10 includes a front facing surface 20, a transverse (radial) or top-facing surface 22, and a rear-facing surface 24. The angled orientation of the front and rear facing surfaces may be useful for directing respective upstream and downstream ultrasonic signals 26, 30 into the fluid flow V. These angled features also promote laminar flow characteristics and resist thrombogenesis. The orientation of the top-facing surface 22 facing transversely (radially) across the internal diameter of the vessel 12 may be useful for directing ultrasonic signals across the vessel interior between the opposing sidewalls 42, 44. As will be described in further detail below, the ultrasonic device 10 may have many different configurations for directing ultrasonic signals in various directions regardless of the surface configuration (e.g., surfaces 20, 22, 24) of the ultrasonic device 10.

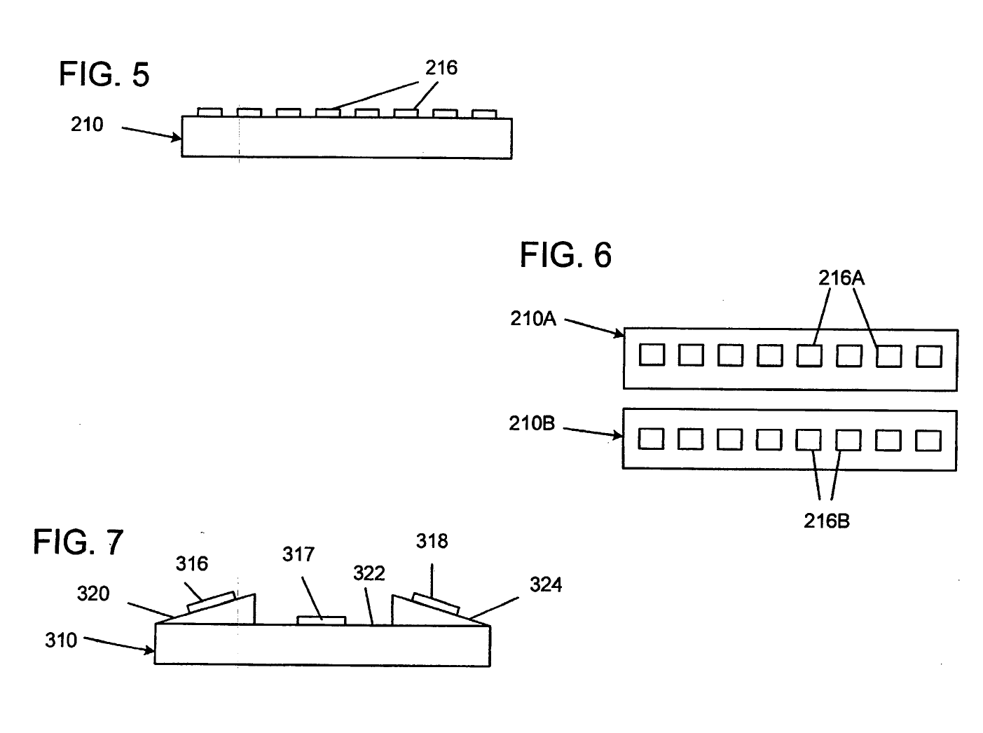

The ultrasonic device 10 may include one or more ultrasound elements such as the transducers shown in FIGS. 5-7 for transmitting and receiving ultrasonic signals. Some types of ultrasound elements are capable of both transmitting and receiving ultrasonic signals, whereas other types of ultrasound elements can either transmit or receive.

In operation, the ultrasonic device 10 is configured to gather and/or determine at least two different types of information. A first type of information gathered or otherwise determined by the ultrasonic device 10 is a velocity v of the fluid flow V. A second type of information determined by the ultrasonic device 10 is the diameter D of the vessel 12. The fluid velocity v together with the diameter D measurement can be used to obtain, through calculations such as integration, a total volume of blood in the vessel over a given distance. This total blood volume is related to a total volumetric flow in the vessel. Accurate monitoring and assessment of volumetric flow in a vessel can be useful for diagnosing and treating many different physiological conditions of a patient. These volumetric measurements combined with the heart rate can be used to calculate cardiac output. The ultrasonic device 10 can provide improved accuracy in determining the volumetric flow, which can result in a more clear and accurate assessment of the patient's physiological conditions.

In order to determine the fluid velocity in the vessel 12, the ultrasonic device 10 first transmits an ultrasonic signal, in a generally upstream or downstream direction (e.g., signals 26, 30). The transmitted signal is reflected off from the fluid flow V reflected ultrasonic signals (e.g., upstream and downstream reflection signals 28, 32) back to the device 10. The reflected signals 28, 32 have a frequency that is altered as a result of reflecting off of the fluid flow V. The difference in frequency between the signals 26, 30 and the signals 28, 32 can be used to calculate the velocity v of the fluid flow V according to Equation 1:

$$v = {f}_{d} \frac{C}{2 {f}_{t} cos θ} \qquad Equation 1$$

Where:
C=velocity of sound
F_{d}=Doppler frequency
Fₜ=frequency transmitted
σ=angle of incidence (see FIG. 1)

The velocity v of the fluid flow V can be determined using either the upstream and reflection signals 26, 28 or the downstream and reflection signals 30, 32. In some embodiments, both the upstream and downstream signal and reflection may be used to determine velocity v to ensure a more accurate velocity determination.

The continuous ultrasound technique will produce difference frequency signals in the tens to hundreds of hertz that are easy to process compared with transit time reflectometry methods. The latter approach generates difference signals in the nanosecond range for velocities of physiological interest. These diminutive time deltas require more complex circuitry to resolve than Doppler frequency shifts.

The vessel diameter D can be determined by a transit time ultrasound signal transmitted radially across the interior of the vessel 12 as signal 34. The transmit signal can be generated intermittently at any desired interval. In one example, the transmit signal is generated at instants that correspond to intervals when the diameter value changes due to systolic and diastolic phases of the cardiac cycle, different activities of the patient (e.g., rest, exercise, etc), a predicted physiological event (e.g., heart failure, fluid imbalance, decompensation events), or to optimize pharmacologic therapy. Cardiac resynchronization therapy (CRT) pacing parameters such as timing delays may be optimized using the cardiac output. This could be combined with a closed feedback loop mechanism providing automaticity for the CRT. The CRT device automatically manipulates pacing parameters until cardiac output is optimized.

The diameter of the vessel can be determined by measuring the time interval required for the signal 34 to reflect signal 36 back to the ultrasonic device 10. Given some known factors such as the speed of sound in a fluid and the approximate internal diameter for the given vessel 12 to which the ultrasound device 10 is attached, the diameter D, and/or related radius values for a given moment in time can be determined with relative accuracy. Transit time ultrasound can be a simple, accurate and reliable means of determining the vessel diameter.

The diameter of a vessel changes over time for a variety of reasons. For example, the diameter of a vessel will change with a change in pressure due to pumping of each heartbeat, which changes the velocity and volumetric flow within the vessel. Typically, a vessel stretches slightly into an increased diameter during systole. Vessels can also expand and contract due to the amount of stress that a patient is experiencing, the type of activity (e.g., sleeping, exercising, etc.) of the patient, body temperature, medications, smoking, alcohol, etc.

The ultrasonic device 10 can be configured to monitor and determine the vessel diameter D at predetermined times of the day and at different intervals regardless of the patient's activities or coordinates with those activities based on other sensed parameters. Each diameter determination can be used separately or an average diameter measurement over a predetermined time period can be determined and used. The ultrasonic device 10 itself or a related system to which the ultrasonic device downloads the data gathered by the device 10 from the signals 26, 28, 30, 32, 34, 36 can perform calculations to provide the desired frequency the vessel diameter D and fluid flow velocity v determinations.

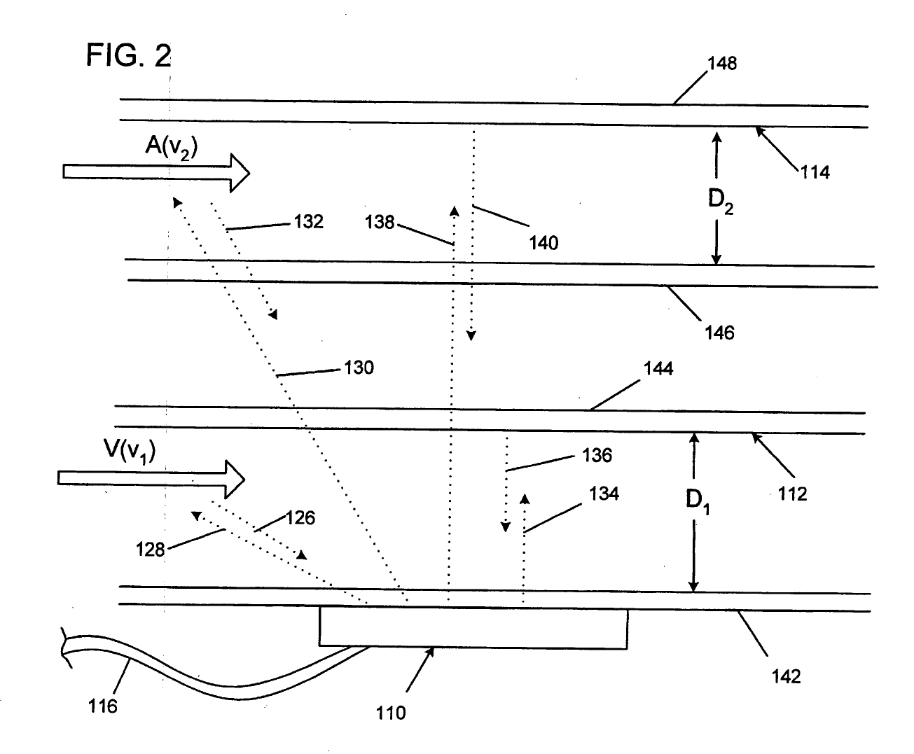

Referring now to FIG. 2, another example ultrasonic device 110 is shown and described with reference to a pair of vessels 112, 114. The ultrasonic device 110 is mounted to an exterior surface of a sidewall 142 of the vessel 112. The ultrasonic device 110 generates a plurality of signals for assessment of a velocity v₁ of fluid flow V within the vessel 112, a diameter D₁ of the vessel 112, a velocity v₂ of fluid flow A in vessel 114, and a diameter D₂ in the vessel 114. FIG. 2 illustrates the versatility of the example ultrasonic device 110 in being able to monitor and assess the fluid velocity v₁ and vessel diameter D₁ of the vessel to which the ultrasonic device 110 is attached, as well as determining similar parameters v₂, D₂ for a vessel extending in close proximity to the vessel to which the ultrasonic device 110 is attached.

The ultrasonic device 110 may include at least one ultrasonic element such as an ultrasonic transducer that generates the ultrasonic signals described below. An upstream signal 126 and first radial signal 130 can be generated by the ultrasonic device 110, and those signals reflected back as upstream reflection signal 128 and first radial reflection signal 136, respectively. The ultrasonic device 110 can also produce a second upstream signal 130 and second radial signal 138 that are reflected back as the second reflected signal 132 and second radial reflected signal 140, respectively. The change in frequency between the signals 126, 128 and the signals 130, 132 can be used to determine the velocities v₁, v₂ of fluid flows V, A. The time delay between the send and receipt of signals 34, 36 and signals 138, 140 can be used to determine the diameters D₁, D₂. In other embodiments, the ultrasonic device 110 can be used to generate downstream oriented signals which can be used in place of or in addition to the upstream signals 126, 128 and 130, 132 for the determination of the fluid flow velocities v₁, v₂.

The reflected signals 126, 132, 136, 140 include amplitude peaks indicative of the various solid objects through which those reflected signals must pass. For example, the reflected signal 132 includes peaks representing the opposing walls 142, 144 of the first vessel 112, as well as a marking indicating the sidewall 146 of the second vessel 114. In the event that the vessels 112, 114 are separated by solid matter (e.g., muscle or fatty tissue) or a fluid having a different density and viscosity from blood (e.g., air or other gas), the reflected signal 132 would include peaks from those different mediums as well. By properly accounting for the various solid objects through which the signals must pass before being received by the device 110, it is possible to determine accurately the desired velocity and diameter measurements for both vessels 112, 114 regardless of the position of device 110.

Because the signals transmitted for velocity determination and the signals transmitted used for diameter determination have different characteristics, the reflected signals will include different types of markings (e.g., amplitude peaks) representing the different mediums through which the signals must pass upon their return to the ultrasonic device 110.

The device 110 may be coupled via a lead 116 to another device. The wired connection can provide a communication means for sending information gathered by the device 110 to a remote location, or may be used to communicate control information from a remote device and the ultrasound device 110. The lead 116 may also be used to power the device 110.

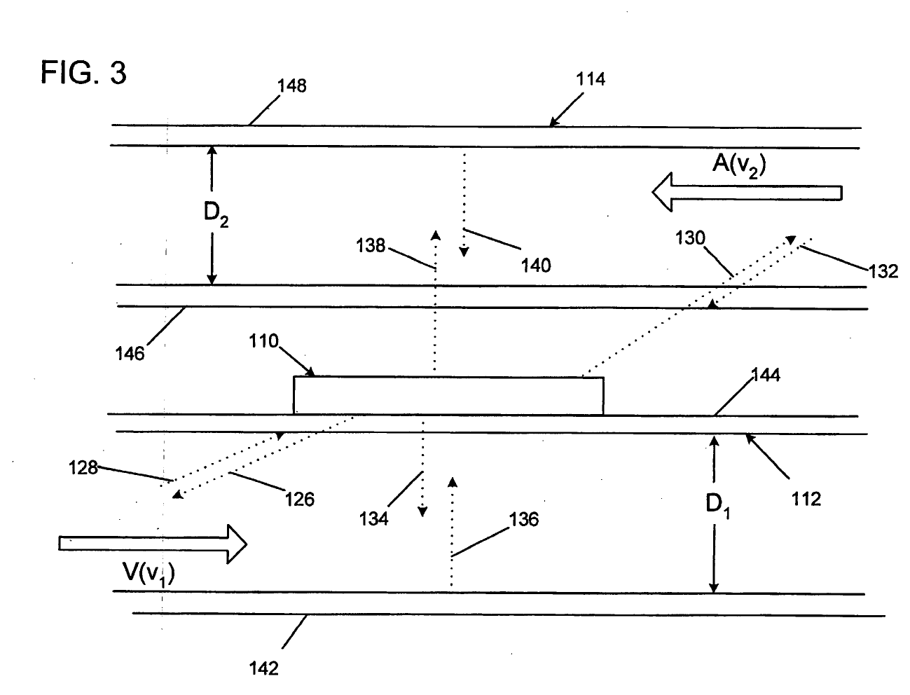

Referring to FIG. 3, a different arrangement of the ultrasonic device 110 is shown. The device 110 is positioned on the exterior surface of the sidewall 144 of vessel 112 and spaced between the vessels 112, 114. The device 110 may include any arrangement of ultrasonic signal generating features positioned on any surface of the device 110 so as to direct the ultrasonic signals in any desired direction. FIG. 3 illustrates device 110 transmitting signal 126 upstream in the vessel 112 and the radial signal 134 radially across the vessel interior 112 toward the sidewall 142. Device 110 also generates an upstream signal 130 in an upstream direction into flow A in vessel 114 (which can be in a direction opposite of the direction of fluid flow in vessel 112) and a radial signal 138 directed radially across the vessel 114 towards the sidewall 148. The signals 126, 134 and 130, 138 may be generated by and directed from ultrasonic elements positioned on either side of device 110. Thus, FIG. 3 illustrates how the ultrasonic device 110 can be configured and oriented to obtain information about different vessels using different orientations and by directing ultrasonic signals in different directions.

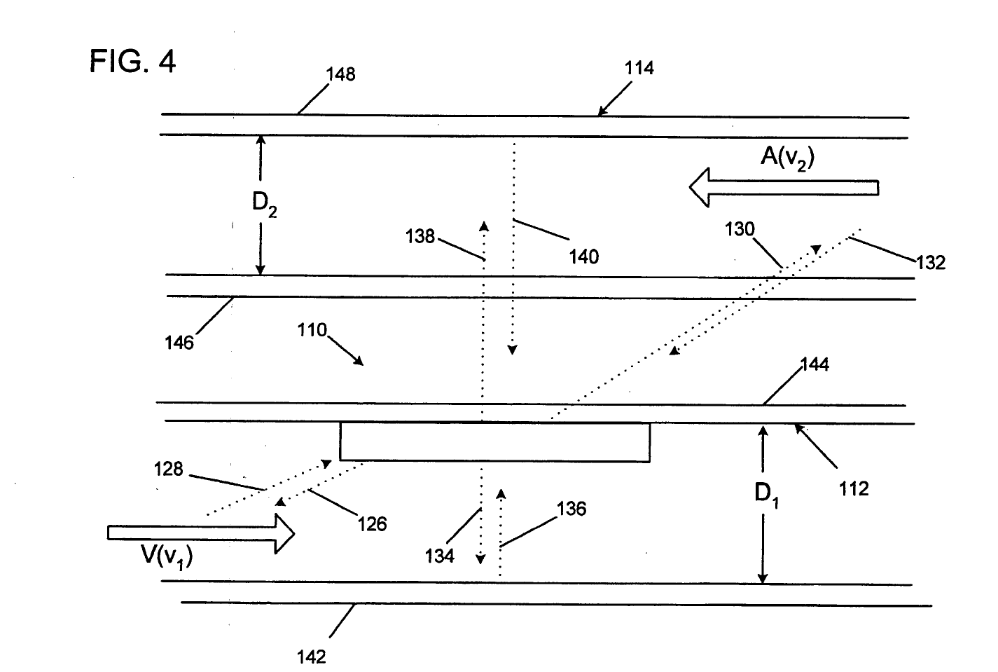

Referring now to FIG. 4, an alternative arrangement for the device 110 is shown relative to the vessel 114. The device 110 is shown mounted to an interior side of the sidewall 144. The device 110 directs ultrasound signals 130 into the blood flow A and collects reflected signals 132 from the second vessel 114. In one example application, the sensor device 110 is placed in the pulmonary artery or superior vena cava but measures blood flow in the adjacent aorta.

FIGS. 5-7 illustrate some alternative ultrasonic device configurations that provide different ultrasonic element arrangements. FIG. 5 illustrates an ultrasonic device 210 having a linear array of ultrasonic elements 216 arranged along the length of the device 210. Each of the elements 216 may be a transmit and receive device or each one may be either a transmit or a receive element. In some arrangements, the transmit and receive elements may be arranged in a side-by-side arrangement, wherein in other embodiments all of the transmit elements may be on one end of the device whereas all of the receive elements are on an opposing end of the device. The ultrasonic elements may be transducers, piezoelectric crystals or other ultrasound signal generating elements.

FIG. 6 illustrates two different devices 210a, 210b aligned adjacent to each other, wherein each device includes a row of elements 216a, 216b, respectively. In this arrangement, the elements 216a may be, for example, transmit elements while the elements 216b may be receive elements, or vice versa. In other embodiments, each row 216a, 216b may have a plurality of transmit and receive elements that are arranged in any desired order, or each element 216a, 216b may have both transmit and receive capabilities. In further embodiments, the elements 216a, 216b may be different types of elements such as Doppler or transmit elements. In some embodiments, the devices 210a, 210b may be combined together into a single device having two rows of elements 216a, 216b.

The arrays of elements 216, 216a, 216b may be, for example, linear array or phased array ultrasonic transducers. Phased array ultrasonic transducers have many capabilities and advantages as described in, for example, U.S. Published Application Nos. 2005/0124880 A1 and 2005/0113700 A1. The capabilities of phased array ultrasonic transducers may be well suited for the example ultrasonic devices disclosed herein for the purpose of obtaining additional and enhanced information that can be useful for monitoring and determining physiological conditions and parameters of a patient. Phased array transducers are typically capable of focusing acoustical energy in a variable orientation beam that can achieve angular displacement of the acoustic energy.

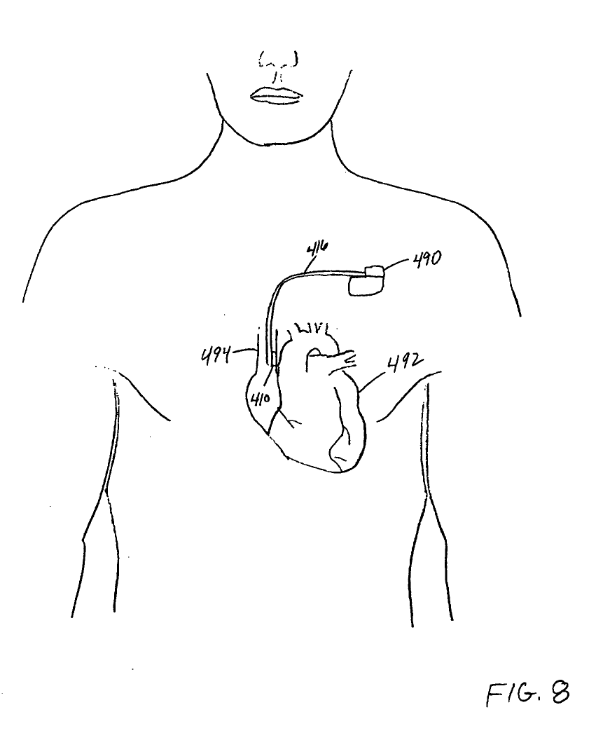

FIG. 7 illustrates another example ultrasonic device 310 that includes front and rear facing surfaces 320, 324, and a radial or top-facing surface 322. Surfaces 320, 322, 324 include respective ultrasonic elements 316, 317, 318. The elements 316, 318 facing either upstream or downstream in a vessel may be Doppler ultrasonic devices whereas the element 317 may be a transmit ultrasonic signal generating element. The surfaces 320, 322, 324 can be configured in many different ways to orient the transducers 316, 317, 318 in any desired direction.

Typically, the ultrasonic devices 10, 110, 210, 310 are generally low profile devices having a greater length than a width dimension. The elongate configuration may provide improved attachment capabilities to an elongate vessel. The low profile feature can provide for reduced flow restriction if the device is positioned within a vessel. The device can include contoured surfaces that match the contoured surfaces of the vessel to provide improved contact with the vessel, reduce fluid flow obstruction if positioned inside of a vessel, and reduce interference with adjacent vessels and organs if positioned outside of a vessel. For devices that are positioned on an exterior of the vessel, the geometry of the device may be less important due to lessened concerns about flow obstruction and other issues such as thrombogenesis, hemolysis, vessel damage, etc.

There are some applications for the inventive principles disclosed herein that may be especially useful. A first example application relates to the embodiments discussed above with reference to FIGS. 2 and 3. In many instances, it is less preferred to provide objects such as inserts, wire leads, implants, and the like within a patient's arteries. Sometimes it is more preferred to provide such objects, if they are required, within a patient's veins if this positioning will provide the same or similar outcome as positioning in an artery. However, arteries in many instances perform functions and the blood carried therein provides information that is important to an assessment of certain physiological conditions. The capability of the ultrasonic devices discussed herein (e.g., see FIGS. 2 and 3) to be mounted on an exterior surface of a targeted vessel (e.g., artery providing the desired information) or to a vessel adjacent to the targeted vessel makes it possible to gather information related to the artery and the blood flow therein without having to position a device inside the artery. As described with reference to FIGS. 2 and 3, some arrangements for the ultrasonic device make it possible to obtain the needed information about the artery and its associated blood flow without physically touching the artery.

The blood flow in the aortic artery can provide important information about a patient's heart because the aorta transports all of the oxygenated blood flow from the heart. Positioning of an ultrasonic device within the aorta near the exit point from the heart may have both advantages and disadvantages. One advantage of using an ultrasonic device at this location in the aorta is that the device can provide information that would lead to an accurate assessment of the stroke volume and cardiac output. Another advantage related to the aorta is that it is easily accessible via other major arteries and is large enough for easier navigation and placement of an ultrasonic device. A disadvantage related to positioning in the aorta may be blood flow obstruction and effects on the blood that is directed to the brain via the carotid arteries, thus increasing the risk of damage to the brain. However the primary issue with this location is the potentially devastating effects of thrombosis.

There are several major veins that extend adjacent to the aorta that can be used as a mounting surface for an ultrasonic device that is capable of gathering information about the aorta and the blood flow in the aorta. The superior vena cava is one example vein that can be used for this purpose since its path to the heart typically extends directly adjacent to the aorta. The superior vena cava or pulmonary artery may be an ideal surrogate vessel to the aorta for positioning of the sensor due to the low pressure and blood flow compared to that in the aorta.

Another important set of arteries that can be monitored with an ultrasonic transducer is the coronary arteries. Like the superior vena cava relationship to the aorta, there are several complimentary veins associated with the coronary arteries that can serve as a mounting surface for an ultrasonic device. By mounting the ultrasonic device to an interior or exterior surface of a coronary vein, the ultrasonic device can be used to assess the coronary artery diameter and the blood flow velocity therein in order to assess important physiologic conditions of the heart.

The example ultrasonic devices discussed herein can be implemented in several different ways. In one example, the ultrasonic device can be mounted to the end of an electrical lead. The lead can be wired to, for example, an implanted medical device (IMD) that is being used solely for controlling the ultrasonic device or is primarily being used for other purposes such as pacing the patient's heart. FIG. 8 illustrates an IMD 490 embedded in a patient and coupled to an ultrasonic device 410 via a lead 416. The device 410 is positioned in a vessel 496 adjacent to the patient's heart 492. In another application, the ultrasonic device is a freestanding unit that communicates wirelessly with an external control device. The wireless communication can take place using, for example, ultrasound, radio frequency (RF), or a magnetic field (induction). The ultrasonic device can be externally powered and charged using similar technologies, which can eliminate the need for an implanted battery or other power source for the ultrasonic device. This may be combined with other sensors such as pressure sensors positioned in the venous side such as the pulmonary artery.

The information gathered by the example ultrasonic devices described above can be useful for diagnosis of a variety of physiologic conditions and can assist in providing more accurate treatment of known physiologic conditions. The accurate assessment of any changes in stroke volume and cardiac output obtained using the ultrasonic device and the amount of change in stroke volume can be useful when optimizing a pacing scheme to maximize cardiac output for a given heart rate. In another example, the cardiac output is useful when optimizing atrium-ventricular delay synchronizing contractions with valve closures). In another example, the rate of change in stroke volume and the cardiac output can be helpful when determining appropriate therapy options. For tachyarrhythmia treatment, if the device senses a tachyarrhythmia, the device may then validate this information with the blood flow to distinguish noise from actual events. This may reduce unnecessary shock therapy due to noise from the lead. In a still further example application, the total cardiac output can be used to determine whether there is sufficient blood flow to sustain consciousness of the patient. Whether or not a patient is conscious can significantly influence the type of treatment applied to a patient by, for example, a hemodynamically based selection of therapy by an IMD (e.g., shock therapy vs. different pacing schemes).

A further purpose for the example ultrasonic devices disclosed herein is to simply monitor trends of the patient's physiological conditions over extended periods of time. This trend information can be logged periodically and evaluated to determine, for example, the patient's general health trend, the patient's physiological response to medications over time, and aggregate health trends for a select group of patients. The ultrasonic device may be a useful component of an Advanced Patient Management System as described in Published Application No. 2004/0127958, Attorney Docket No. 13569.33US01, filed Dec. 27, 2002, and entitled “Advanced Patient Management System Including Interrogator/Transceiver Unit,” which application is incorporated herein by reference.

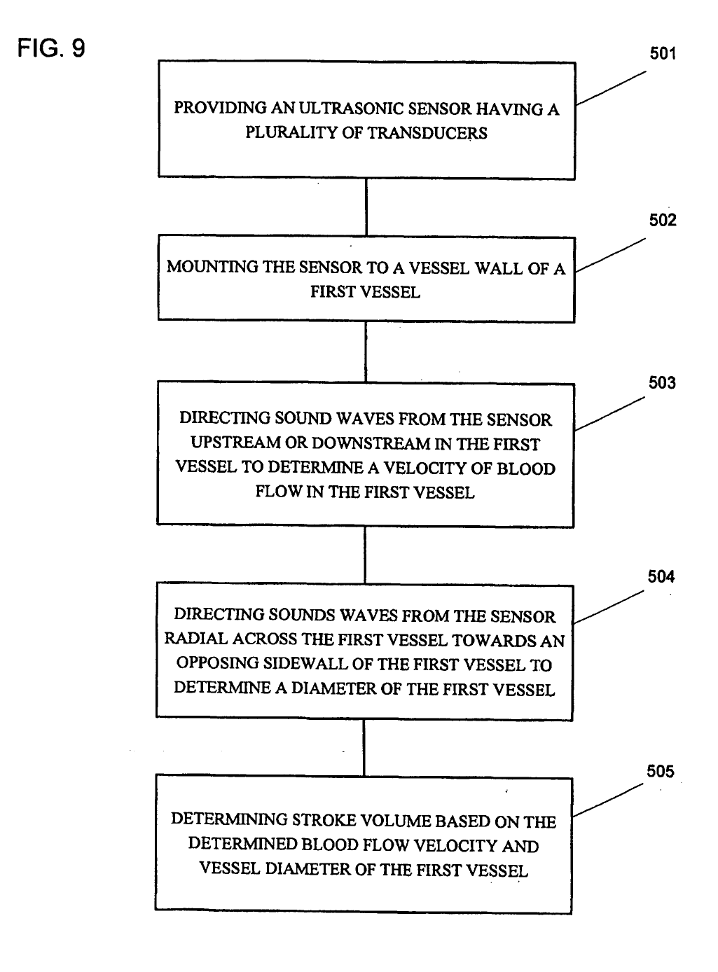

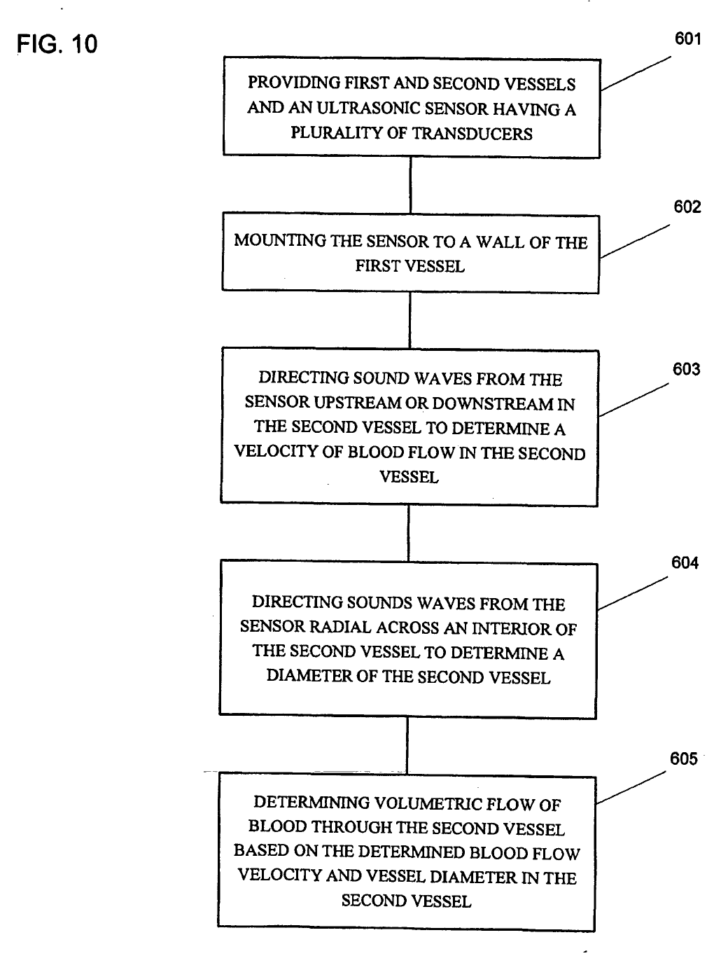

Referring to FIGS. 9 and 10, some example methods related to monitoring a patient's heart and determining output from the heart are shown and described. Details related to the illustrated steps 501-505 and 601-601 in FIGS. 9 and 10 are described with reference to FIGS. 1-8 above.

The above specification, examples and data provide a complete description of the manufacture and use of the composition of the invention. Since many embodiments of the invention can be made without departing from the scope of the invention, the invention resides in the claims hereinafter appended.

We claim:

1. An ultrasonic implantable device, comprising:
an ultrasonic sensor having a plurality of transducers, the sensor being mountable to a vessel wall of a vessel, a first of the transducers configured to direct sound waves in a direction at least partially upstream or downstream in the vessel, and a second of the transducers configured to direct sound waves in a transverse direction through an interior of the vessel against a distal portion of the vessel;
wherein the sensor monitors a change in frequency of the sound waves from the first transducer to determine a fluid velocity in the vessel, and the sensor monitors a reflection time of the sound waves from the second transducer that return from the distal portion of the vessel wall to determine an internal diameter of the vessel.

2. The implantable device of claim 1, further comprising an electronic wire lead electronically coupled to the sensor and coupleable to an implanted medical device (IMD).

3. The implantable device of claim 1, wherein the plurality of transducers include piezoelectric crystals arranged as a phased array.

4. The implantable device of claim 1, wherein the sensor includes a first transducer mounting surface facing at least partially upstream or downstream in the vessel, and a second transducer mounting surface facing across the interior of the vessel, wherein at least one of the transducers is positioned on each of the first and second transducer mounting surfaces.

5. The implantable device of claim 1, wherein the determined fluid velocity and the determined internal vessel diameter are used to determine a volumetric flow rate in the vessel.

6. A method of monitoring heart performance, the method comprising:
mounting an ultrasonic sensor having a plurality of transducers to a vessel wall of a first vessel;
directing sound waves from the sensor upstream or downstream in the first vessel to determine a velocity of blood flow in the first vessel;
directing sounds waves from the sensor across the first vessel towards an opposing sidewall of the first vessel to determine a diameter of the first vessel; and
determining blood stroke volume of the heart based on the determined blood flow velocity and the diameter of the first vessel.

7. The method of claim 6, further comprising determining cardiac output by multiplying stroke volume by a heart rate of the heart.

8. The method of claim 6, further comprising determining cardiac output by measuring and combining both the stroke volume and information about a heart rate of the heart.

9. The method of claim 8, wherein the information about a heart rate is obtained from electrogram data that is received from an implanted medical device.

10. The method of claim 8 wherein the information about a heart rate is obtained from electrodes integrated into the sensor.

11. The method of claim 8 wherein the information about a heart rate is obtained from the periodicity of the velocity signal.

12. The method of claim 8 wherein the information about a heart rate is obtained from the periodicity of the diameter signal.

13. The method of claim 6, wherein the sensor is mounted to an interior surface of the vessel wall.

14. The method of claim 6, wherein the sensor is mounted to an exterior surface of the vessel wall.

15. The method of claim 6, wherein determining the blood flow in the vessel includes monitoring a change in frequency of the sound waves directed upstream or downstream in the vessel.

16. The method of claim 6, wherein determining the vessel diameter includes monitoring a reflection time of the sound waves reflected from the opposing sidewall of the vessel.

17. The method of claim 6, further comprising:
directing sound waves from the sensor upstream or downstream in a second vessel located near the first vessel to determine a blood flow velocity in the second vessel;
directing sound waves from the sensor radially across an interior of the second vessel to determine a diameter of the second vessel; and
determining a blood volume flow rate in the second vessel based on the determined blood flow velocity and vessel diameter of the second vessel.

18. The method of claim 17, wherein the first vessel is a vein and the second vessel is an artery.

19. The method of claim 6, further comprising:
directing sound waves from the sensor upstream or downstream in the heart to determine a blood flow velocity of blood in a chamber of the heart, wherein the heart is located near to the first vessel;
directing sound waves from the sensor transversely across an interior of the heart chamber to determine a diameter of the heart chamber; and
determining a blood volume flow rate in the heart chamber based on the determined blood flow velocity and vessel diameter of the heart chamber.

20. The method of claim 6, wherein the first vessel is selected from the group consisting of a coronary artery, a coronary vein, a pulmonary artery, a pulmonary vein, a superior vena cava vein, and the aorta.

21. The method of claim 6, further comprising electrically coupling the sensor to the end of an electric lead.

22. The method of claim 6, further comprising powering the sensor remotely with a wireless connection.

23. The method of claim 6, further comprising conducting wireless communicating between the sensor and a remote device.

24. The method of claim 6, further comprising combining output from one or more additional sensors with output from the ultrasonic sensor to provide greater sensitivity in monitoring a decompensation event, the one or more additional sensors selected from a group comprising a heart rate sensor, a pressure sensor, a temperature sensor, an accelerometer, and an acoustical sensor.

25. The method of claim 6, wherein information provided by the ultrasonic sensor is used in a closed feedback loop system that monitors physiologic performance and is coupled with a therapeutic device to deliver a therapy according to pre-programmed algorithms.

26. The method of claim 6, wherein information from the ultrasonic sensor is used to determine peak flow rate as a cardiac contractility measurement similar to dP/dt assessments and is used for therapeutic optimization purposes.

27. A method of monitoring blood flow in a first vessel, the method comprising:
mounting an ultrasonic sensor having a plurality of transducers to a wall of a second vessel;
directing sound waves from the sensor upstream or downstream in the first vessel to determine a velocity of blood flow in the first vessel;
directing sound waves from the sensor radial across an interior of the first vessel to determine a diameter of the first vessel; and
determining volumetric flow of blood through the first vessel based on the determined blood flow velocity and the determined diameter of the first vessel.

28. The method of claim 27, wherein the first vessel is an artery and the second vessel is a vein, the artery and the vein located adjacent to an intervening vessel.
